(19) United States
(12) Patent Application Publication
(10) Pub. No.: US 2007/0185053 A1
(43) Pub. Date: Aug. 9, 2007

(54) SINGLE-DOSE NEEDLE-FREE ADMINISTRATION OF ANTITHROMBOTIC MEDICATIONS

(75) Inventor: Lawrence Linn, Walnut Creek, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Zogenix, Inc.

(21) Appl. No.: 11/624,076

(22) Filed: Jan. 17, 2007

Related U.S. Application Data
(60) Provisional application No. 60/759,862, filed on Jan. 17, 2006.

(51) Int. Cl. A61K 31/727; A61M 31/00

(52) U.S. Cl. 514/56; 604/500

(57) ABSTRACT

An antithrombotic drug is administered by a needle-free injection device for prevention or treatment of venous thromboembolism. In an embodiment, the needle-free injection device is a pre-filled, single-use, disposable device with antithrombotic medication and labeled regarding drug, dose and time of delivery.

### CROSS-REFERENCE

This application claims the benefit of U.S. Provisional Application No. 60/759,862, filed Jan. 17, 2006, which application is incorporated herein by reference.

### FIELD OF THE INVENTION

The present invention relates to needle-free delivery of pharmaceutical compositions containing antithrombotic medications as an active ingredient and to methods of prophylaxis (prevention) of thromboembolic (blood clotting) disorders.

### BACKGROUND OF THE INVENTION

The antithrombotic class of drugs are used to treat or prevent the formation of blood clots, or thrombi, in the body. Thrombosis is a naturally occurring physiologic process. Under normal circumstances, a physiologic balance is maintained between factors that promote and retard blood coagulation. A disturbance in this equilibrium may trigger a coagulation event to occur at a time or to an extent that leads to increase risk of tissue damage, symptomatic pain, or even death.

Venous thromboembolism, or VTE, is a collective term that refers to an occlusive blood clot, or thrombus, in a vein. Deep vein thrombosis (DVT) and pulmonary embolism (PE) represent different manifestations of VTE. Clots that occur in deep veins such as in the leg or clots that occur in or travel to the lungs from other areas of the body are serious medical conditions that require acute treatment intervention when detected or prevention in conditions known to increase the risk of thrombi formation, such as after surgery or during extended periods of immobilization (e.g., confinement to a bed).

In the U.S. complications from DVT kill up to 200,000 people a year U.S. See e.g., Turpie A G G, et. al., N Engl J Med 1986 315:925-929; Hull R D, et. al., JAMA 1990 263:2313-2317; Lassen M R, et al., Acta Orthop Scand 1991 62:33-38; Hoek J A, et al., Thromb Haemost 1992 67:28-32; Cohen S H, et al., J Bone Joint Surg Am 1973 55:106-112; Stullberg B N, et al., J Bone Joint Surg Am 1984 66:194-201; Lynch A F, et al., J Bone Joint Surg Am 1988 70:11-14; Stringer M D, et al., J Bone Joint Surg Br 1989 71:492-497; Eriksson B I, et al., J Bone Joint Surg Am 1991 73:484-493; Mohr D N, et al., Mayo Clin Proc 1992 67:861-870; Warvick D, et al., J Bone Joint Surg Br 1995 77:6-10; Murray D W, et. al., J Bone Joint Surg B r 1996 78:863-870; Geerts W, et al., Chest 2001 119(1Suppl):132S-175S; Wells P S, et al., Ann Intern Med 1995 122:47-53; Leclerc J R, et al., Arch Intem Med 1998 158:873-878.

Many pharmacologic agents are currently available to prevent thrombosis. Agents that retard or inhibit the process belong to the class of anticoagulants. Agents that prevent the growth or formation of thrombi are termed antithrombotics and include anticoagulants and antiplatelet drugs, whereas thrombolytic drugs break up existing thrombi.

Compounds which act as antithrombotic agents have been described in U.S. Pat. Nos. 5,332,822; 5,492,895; 5,612,363, 5,691,364; 5,693,641; 5,721,214; 5,726,173; 5,753,635; 5,846,970; 5,849,759; 5,889,005; 6,107,280; 6,140,351; 6,150,329; 6,180,627; 6,200,976; 6,242,432; 6,248,770; 6,271,215; 6,280,731; 6,287,794; 6,300,330; 6,300,342; 6,333,338; 6,395,731; 6,417,203; 6,432,955; 6,444,672; 6,451,832; 6,458,793; 6,486,129; 6,500,803; 6,583,173; 6,599,881; 6,723,723; 6,730,672; 6,753,331; 6,774,110; 6,797,710; and 6,924,296 incorporated to disclose and describe antithrombotic agents, as well as formulations and uses thereof.

Standard unfractionated hepalin (UFH) is a widely used anticoagulant modality. UHF has been used for this purpose in various forms since its discovery by McLean in 1916. UHF acts in conjunction with a circulating plasma cofactor, ATIII and, in its presence, catalyzes the inactivation of factors IIa, Xa, IXa, and XIIa. By inactivating thrombin, heparin not only prevents fibrin formation but also inhibits thrombin-induced activation of factor V and factor VIII. Of these, factors IIa and Xa are most sensitive. Therefore, heparin has both anticoagulant and antithrombotic properties.

Heparin is a heterogeneous mixture of molecules that contain a range of molecular weights of 3,000-30,000, with an average of approximately 15,000. Only one third of the heparin molecules have an active binding site for ATIII, and this fraction is responsible for most of the anticoagulant activity. Heparin is effective when given by intravenous (IV) or subcutaneous (SC) administration but is inactivated in the GI tract. Heparin has a rapid onset of action, its half-life is brief in comparison to warfarin, and it binds to platelets, endothelial cells, and macrophages in vivo. Therapeutic levels of heparin are measured by propensity towards clotting using the activated partial thromboplastin time (aPTT) test. Because of the rapid clearance of heparin from the bloodstream, therapeutic levels are more likely achieved with continuous IV infusion.

Disadvantages of heparin therapy include variable pharmacokinetics, the requirement for aPTT monitoring for adjusted-dose regimens, short half-life and low bioavailability, and lack of an oral dosage form due to inactivation in the gut (although an oral form has been tested in clinical trials). In addition, a small percentage of patients (2-4%) are susceptible to the development of heparin-induced thrombocytopenia (HIT), which is an antibody-mediated adverse reaction that can cause venous and arterial thrombosis.

Low molecular weight heparins (LMWHs) can either be derived from standard UFH or synthesized chemically. LMWHs are derived when standard UFH is treated by a variety of enzymatic or chemical methods to select those lower molecular weight moieties that contain the active ATIII binding site. Alternatively, a class of antithrombotics can be produced entirely by chemical synthesis, such as is done for the anti-factor Xa pentasaccharide enoxaparin and others in the synthetics class. The average molecular weight of fractionated heparin is 4,500 in comparison to the usual 15,000. The molecular weight threshold under which anti-factor Xa activity is maximized is 5,400 Daltons (Da).

In fractionating standard heparin, the polysaccharide side chain of the heparin molecule is decreased from 18 Units (U) to approximately 13 U. As the length of the side chain is decreased, the ability of the molecule to prolong the aPTT is lost, but the ability to complex with ATIII is retained. LMWHs, whether derived or synthetic, do not require monitoring with aPTT testing.

The pharmacologic effect of this transformation is to make the LMWH more bioavailable (approximately 90%, compared with 29% for UFH) and to lengthen its half-life to 4 hours from 1 hour for UFH. LMWH also increases the activity ratio of anti-Xa to anti-IIa, resulting in increased antithrombotic activity. In experimental models and animal studies, LMWH produces less microvascular bleeding than UFH, but this finding has not been duplicated in human trials. Compared to placebo, LMWHs produced a 70-80% risk reduction for DVT in numerous studies without an increase in major bleeding in high-risk orthopedic patients. Meta-analysis comparison with a variety of other methods of DVT prophylaxis, including low-dose UFH, adjusted-dose heparin, and warfarin, have demonstrated improvement in DVT prophylaxis without increase in hemorrhagic complications.

LMWHs currently approved for use include: enoxaparin (Lovenox®, Aventis), dalteparin (Fragmin®, Pfizer), nadroparin (Fraxiparine®, GlaxoSmithKline), tinzaparin (Innohep®, Dupont), ardeparin (Normiflo®, Wyeth-Ayerst), reviparin (Clivarine®, Abbott), danaparoid (Orgaran®, Organon), fondaparinux sodium (Arixtra®, GlaxoSmithKline).

Current treatment recommendations provide that LMWH (or other prophylaxis medication), after treatment initiation shortly following surgery, should be continued for 10 days after knee replacement surgery and for 3 weeks following hip replacement surgery. Administration is by once or twice daily subcutaneous injection. In high-risk patients, prophylaxis should last 30-40 days or longer postoperatively. This therapy can reduce total and proximal DVT by at lease 50% without increasing major bleeding events. Patients with a history of prior DVT or PE and those who have inherited thi-ombophilia may require even longer treatment.

Following patient discharge from the hospital, most people can be treated with LMWH while at home. Many patients, however, are needle-averse or suffer from needle-phobia and ensuring 100% treatment compliance can be problematic, especially due to the increased risk of thrombosis resulting from even one missed dose of an antithrombotic. In addition, it is a problem that patients need to be trained to self administer an injection, although the number of injections they would self administer is only a few. In addition, a needle and syringe in general needs to be filled, which further complicates self administration and reduces compliance.

Needle-free injectors are available using many different types of energy, and the energy may be supplied by the user, for example where a spring is manually compressed and latched to temporarily store the energy until it is required to “fire” the injector. Alternatively, the injector may be supplied having the energy already stored—for instance by means of a precompressed spring (mechanical or gas), or by pyrotechnic charge.

Some injectors are intended for disposal after a single use, whereas others have a re-loadable energy storage means and a disposable medicament cartridge, and there are many combinations to suit particular applications and markets. For the purposes of the present disclosure, the term “actuator” will be used to describe the energy storage and release mechanism, whether or not it is combined with the medicament cartridge. In all cases, it is necessary to arrange for sufficient force at the end of the piston stroke to deliver the entire medicament at the required pressure: if a spring is used, this is called “pre-loading”.

EP 0 063 341 and EP 0 063 342 disclose a needle-free injector which includes a piston pump for expelling the liquid to be injected, which is driven by a motor by means of a pressure agent. The liquid container is mounted laterally to the piston pump. The amount of liquid required for an injection is sucked into the pump chamber by way of an inlet passage and a flap check valve when the piston is retracted. As soon as the piston is moved in the direction of the nozzle body the liquid is urged through the outlet passage to the nozzle and expelled. The piston of the piston pump is a solid round piston.

EP 0 133 471 describes a needle-free vaccination unit which is operated with carbon dioxide under pressure, from a siphon cartridge by way of a special valve.

EP 0 347 190 discloses a vacuum compressed gas injector in which the depth of penetration of the injected drug can be adjusted by means of the gas pressure and the volume of the drug can be adjusted by way of the piston stroke.

EP 0 427 457 discloses a needle-free hypodermic syringe which is operated by means of compressed gas by way of a two-stage valve. The injection agent is disposed in an ampoule which is fitted into a protective casing secured to the injector housing. The ampoule is fitted on to the end of the piston rod. Disposed at the other end of the ampoule is the nozzle whose diameter decreases towards the end of the ampoule.

WO 89/08469 discloses a needle-free injector for one-off use. WO 92/08508 sets forth a needle-free injector which is designed for three injections. The ampoule containing the drug is screwed into one end of the drive unit, with the piston rod being fitted into the open end of the ampoule. At its one end, the ampoule contains the nozzle through which the drug is expelled. A displaceable closure plug is provided approximately at the center of the length of the ampoule. The dose to be injected can be adjusted by changing the depth of the ampoule. The piston rod which projects from the drive unit after actuation of the injector is pushed back by hand. Both units are operated with compressed gas.

WO 93/03779 discloses a needle-free injector with a two-part housing and a liquid container which is fitted laterally to the unit. The drive spring for the piston is stressed by means of a drive motor. The spring is released as soon as the two parts of the housing are displaced relative to each other by pressing the nozzle against the injection location. Respective valves are provided in the intake passage for the liquid and in the outlet of the metering chamber.

WO 95/03844 discloses a further needle-free injector. It includes a liquid-filled filled cartridge which at one end includes a nozzle through which the liquid is expelled. At the other end the cartridge is closed by a cap-type piston which can be pushed into the cartridge. A piston which is loaded by a prestressed spring, after release of the spring, displaces the cap-type piston into the cartridge by a predetermined distance, with the amount of liquid to be injected being expelled in that case. The spring is triggered as soon as the nozzle is pressed sufficiently firmly against the injection location. This injector is intended for one-off or repeated use. The cartridge is arranged in front of the spring-loaded piston and is a fixed component of the injector. The position of the piston of the injector which is intended for a plurality of uses is displaced after each use by a distance in a direction towards the nozzle. The piston and the drive spring cannot be reset. The pre stressing of the spring is initially sufficiently great to expel the entire amount of liquid in the cartridge all at once. The spring can only be stressed again if the injector is dismantled and the drive portion of the injector assembled with a fresh, completely filled cartridge.

U.S. Pat. No. 5,891,086 describes a needle-free injector, combining an actuator and a medicament cartridge. The cartridge is pre-filled with a liquid to be injected in a subject, and having a liquid outlet and a free piston in contact with the liquid, the actuator comprising an impact member urged by a spring and temporarily restrained by a latch means, the impact member being movable in a first direction under the force of the spring to first strike the free piston and then to continue to move the piston in the first direction to expel a dose of liquid through the liquid outlet, the spring providing a built-in energy store and being adapted to move from a higher energy state to a lower energy state, but not vice versa. The actuator may comprise trigger means to operate the said latch, and thus initiate the injection, only when a predetermined contact force is achieved between the liquid outlet of the said cartridge and the subject.

In U.S. Pat. No. 3,859,996, Mizzy discloses a controlled leak method to ensure that the injector orifice is placed correctly at the required pressure on the subject's skin at the corrrect normal to the skin attitude. When placement conditions are met, controlled leak is sealed off by contact pressure on the subject's skin, the pressure within the injector control circuit rises until a pressure sensitive pilot valve opens to admit high pressure gas to drive the piston and inject the medicament.

In WO Patent 82/02835, Cohen and Ep-A-347190 Finger, disclose a method to improve the seal between the orifice and the skin and prevent relative movement between each. This method is to employ a vacuum device to suck the epidermis directly and firmly onto the discharge orifice. The discharge orifice is positioned normal to the skin surface in order to suck the epidermis into the orifice. This method for injection of the medicament into the skin and the injector mechanism are different and do not apply to the present invention because of its unique ampule design.

In U.S. Pat. No. 3,859,996 Mizzy discloses a pressure sensitive sleeve on the injector which is placed on the subject, whereby operation of the injector is prevented from operating until the correct contact pressure between orifice and the skin is achieved. The basic aim is to stretch the epidermis over the discharge orifice and apply the pressurized medicament at a rate which is higher than the epidermis will deform away from the orifice.

In U.S. Pat. No. 5,480,381, T. Weston discloses a means of pressuring the medicament at a sufficiently high rate to pierce the epidermis before it has time to deform away from the orifice. In addition, the device directly senses that the pressure of the discharge orifice on the subject's epidermis is at a predetermined value to permit operation of the injector. The device is based on a cam and cam follower mechanism for mechanical sequencing, and contains a chamber provided with a liquid outlet for expelling the liquid, and an impact member, to dispell the liquid.

In U.S. Pat. No. 5,891,086, T. Weston describes a needle-free injector that contains a chamber that is pre-filled with a pressurized gas which exerts a constant force on an impact member in order to strike components of a cartridge and expulse a dose of medicament. This device contains an adjustment knob which sets the dose and the impact gap, and uses direct contact pressure sensing to initiate the injection.

### SUMMARY OF THE INVENTION

A liquid formulation of an antithrombotic medicament in a needle-free injection format is a convenient, patient preferred method of self-administering the antithrombotic in individuals who are at risk of developing VTE following abdominal surgery, hip replacement, hip fracture, or knee replacement surgery. Following discharge from the hospital setting, patients are typically advised to self-inject for approximately 10 days, but treatment periods may be longer in patients who are at greater risk of clotting, such as those who remain immobilized and/or who have a history of VTE. Injection with a needle-free device results in less patient apprehension relative to injection by needle, improved treatment compliance, lower patient morbidity and mortality, and requires less training compared to conventional needle and syringe.

Methods and formulations for the therapeutic administration of an antithrombotic medicament are disclosed. In particular, the subject methods provide for the administration of different dosage strengths of an antithrombotic medication, using a needle-free drug delivery system which is designed to provide pharmacokinetics which are bioequivalent to a conventional needle or syringe product.

An aspect of the invention is that the antithrombotic will be provided in a range of formulation dosage strengths for single-dose injection by the needle-free injector for the treatment and prophylaxis of different medical conditions.

Another aspect of the invention is that the antithrombotic is delivered by needle-free injection to provide prophylaxis (i.e., prevention) of venous thromboembolism including deep vein thrombosis and pulmonary embolism in patients undergoing treatments including, but not limited to, hip replacement, knee replacement, hip fracture, or abdominal surgery.

Another aspect of the invention is that the antithrombotic medication is delivered by needle-free injection to treat emergent acute venous thromboembolism or deep vein thrombosis in patients while or after clot formation is occurring or has already occurred.

Another aspect of the invention is to provide a simple device allowing patients to self-administer antithrombotics following medical procedures including surgeries, but not limited to, hip replacement, knee replacement, hip fracture, or abdominal surgery, without requiring the patient learn how to self-administer an injection using a needle and syringe.

Another aspect of the invention is to provide a pre-filled disposable injection device to simplify self-administration of antithrombotics following medical procedures including, but not limited to, hip replacement, knee replacement, hip fracture, or abdominal surgery.

Another aspect of the invention is a kit comprised of a needle-free injector device and a plurality of ampules wherein each of the ampules hold an antithrombotic drug and are labeled with respect to a characteristic chosen from a type of drug, amount of drug, day of the week, time, and all or any of these characteristics.

Another aspect of the invention is a kit comprised of a plurality of a disposable needle-free injector devices wherein each of the devices is preloaded with a liquid formulation comprised of a pharmaceutically acceptable injectable carrier and a pharmaceutically active antithrombotic agent and wherein each device is labeled with a characteristic chosen from type of drug, amount of drug, day, time, and all or any of these characteristics wherein the kit further includes instructions for the use of the needle-free injection device.

These and other aspects, objects, advantages, and features of the invention will become apparent to those persons skilled in the art upon reading the details of the methodology as more fully described below.

### DETAILED DESCRIPTION OF THE INVENTION

Before the present method and device is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a drug” includes a plurality of such drug and reference to “the surgery” includes reference to one or more surgeries and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “antithrombotic” or “antithrombotic drugs” refers to any compound intended to treat or prevent the formation of a blood clot particularly in a human.

The term “bioequivalent” refers to a finding of pharmacokinetic equivalence between methods of administration of a drug formulation or equivalence between different drug formulations to a human subject wherein several key pharmacokinetic variables, or parameters, are analyzed and compared and between a test and a reference product and found to be equivalent within specified criteria. This is done by calculation of the area under the blood concentration versus time profile (see definition below) to the last blood sampling time-point and by extrapolation of the profile to infinity (AUCt and AUCinf, respectively), and by calculation of the maximal concentration and the time of maximal concentration after dose administration from the blood concentration versus time profile (Cmax and Tmax, respectively). Bioequivalence may be defined as having been demonstrated if the 90% confidence intervals of the log-transformed ratios of AUCt, AUCinf, and Cmax are found to be within the acceptance range convention of 80%-125%. The additional pharmacokinetic parameter of Tmax may be included in the definition of bioequivalence wherein the 90% confidence intervals of the ratios of Tmax for the test product are within the range of 80%-125% of the reference product.

The term “blood concentration versus time profile” shall be interpreted to mean the concentration of a drug in the blood or plasma over time. This can be characterized by means of a graph showing the concentration of a drug (e.g., antithrombotic agent) on the Y-axis and time on the X-axis.

The term “carrier” shall mean a non-active portion of a formulation which may be a liquid which may act as a solvent. In aqueous formulations, it is a liquid, flowable, pharmaceutically acceptable excipient material which an antithrombotic is suspended in or more preferably dissolved in. Useful carriers do not adversely interact with the antithrombotic and have properties which allow for delivery by injection. Preferred carriers for liquid solutions include water, saline, and mixtures thereof. Other carriers can be used provided that they can be formulated to create a suitable solution and do not adversely affect the antithrombotic thereof or human tissue.

The term “DVT” or “Deep Vein Thrombosis” refers to the formation of a blood clot (solid mass of blood) within a blood vessel—particularly the large deep veins of the lower legs. They can be caused by a prolonged lack of body movement. The clot can block the flow of blood through the vessel and if the clot stays, it can cause tissue damage, due to lack of oxygen.

The term “liquid formulation” is used herein to describe any pharmaceutically active form of an antithrombotic drug by itself or with a pharmaceutically acceptable carrier in flowable liquid form for administration by the needle-free injector. Such formulations are preferably solutions, e.g., aqueous solutions, saline solutions, and colloidal suspensions. Formulations can be solutions or suspensions of drug in any fluid including fluids in the form of a low boiling point propellant. Suspensions can include colloids, emulsions, nano-suspensions, and the like.

The term “excipient” is used to refer to any substance, including a carrier, added to an active drug substance to permit the mixture to achieve the appropriate physical characteristics necessary for effective delivery of the active drug.

The term “PE” or “Pulmonary Embolism” refers to the lodging of a blood clot in the lumen (open cavity) of a pulmonary artery, causing a severe dysfunction in respiratory function. Pulmonary emboli often originate in the deep leg veins and travel to the lungs through blood circulation. Symptoms include sudden shortness of breath, chest pain (worse with breathing), and rapid heart and respiratory rates.

The term “prophylaxis” is used to refer to the administration of a drug used to prevent the occurrence or development of an adverse condition or medical disorder.

The term “spring” is used to refer to any method capable of storing energy for use in propelling the medicament in the syringe into and through the patient's skin and into body. This method may be mechanical, as by a compressible metal component wherein the stored energy is released within the syringe and propels the drug by a mechanism into the patient's body, or may be by a compressed gas which in which the energy is stored and then is then released and expands at a rapid rate of decompression thereby injecting the medicament through the patient's skin and into the body.

The term “VTE”or “Venous Thromboemobolism” refers to the formation of a blood clot in the veins which can travel from the site where it formed and block blood flow at another location.

### Invention in General

The invention includes a device for the delivery of an antithrombotic medication. The device is comprised of a needle-free injector and a liquid formulation which is comprised of a pharmaceutically acceptable carrier and a pharmaceutically active antithrombotic agent. Specific agents are described here.

The invention also includes methods of treatment which comprises screening patients with respect to factors relating to injection with needles including the patient's fear of needles and the patient's ability to use needles. The method includes providing the patient with a screened criteria such as fear of needles or inability to use a needle injector with a needle-free injector device which includes a liquid formulation having an antithrombotic agent therein and then instructing the patient with respect to the use of the device. The invention also includes kits which are comprised of needle-free injectors and a plurality of ampules (which are labeled as described here) which are loaded with an antidthrombotic compound. Other kits can include a plurality of single use disposable needle-free injector devices which are loaded with a formulation comprising an antithrombotic drug.

An aspect of the invention is to provide greater convenience to post operative patients in need of treatment with antithrombotic medication. To provide greater convenience the patient can be provided with a kit of the invention which kit is comprised of a needleless injector device and antithrombotic medication which can be administered from the device. In one embodiment of the kit the device is present with a plurality of ampoules of antithrombotic medication such as 3 or more, 5 or more, or 10 or more ampoules of medication wherein each of the ampoules of medication is labeled with an appropriate label which may include the name of the medication, the amount of dose of medication, and the time when the medication should be administered which can include the time of day and day of week or month and date when the medication is to be delivered. In another embodiment of the invention the kit provided to the patient includes a plurality of needleless injector devices wherein each of the devices is a one use, disposable device preloaded with an antithrombotic medication. Each device is also labeled with a label as indicated above with respect to the ampoules. By providing the patient with a kit comprised of a plurality of ampoules or a plurality of disposable needleless injector devices which are labeled the patient can more conveniently administer antithrombotic medication particularly in a post operative situation where the patient is unfamiliar with the use of needles.

In order to further understand the invention in detail antithrombotic medications, needle-free injector devices and methods of self-administration of drugs are described below.

### Antithrombotic Medications

The invention provides for injectible formulations of antithrombotic medications for use in a needle-free injection device.

The total dosage of the antithrombotic medication for the prophylaxis or acute treatment of VTE (including DVT and PE) will be that which provides bioequivalent systemic dose (±20%) or (±10%) or (±5%) available from the corresponding recommended dosage to be administered by conventional needle and syringe injection, depending on the surgical or medical condition requiring VTE prophylaxis.

A range of dosage formulations, not limited to but most commonly comprising and dependent upon the particular antithrombotic medication, from 1 mg to 40 mg and up to 1,000 mg of an antithrombotic in a solution with a volume ranging from 0.1 mL to 10 mL, preferably between 0.2 mL to 5 mL, and most preferably between 0.5 mL to 2.0 mL. These formulations allow treatment for the prophylaxis of VTE following hip or knee replacement surgery, abdominal surgery, or during severely restricted mobility conditions, or for treatment of acute emergent VTE after or while the condition is occurring.

Formulations of the invention are in the form of a sterile solution of the active pharmaceutical agent using water for injection and/or other suitable liquid solvents, carriers, or excipients to achieve the dosage strengths necessary to comprise a total needle-free injector fill volume between 0.1 mL to 10 mL. Other components may be added to enhance or maintain chemical stability, including buffers, chelating agents, protease inhibitors, isotonic modifiers, and the like.

### Needle-Free Injection

The invention provides that the antithrombotic drug be delivered from a needle-free injection device. Many different needle free injectors can be used, including but not limited to devices sold under the following trademark names PenJet®, Iject®, J-Tip®, CrossJect®, Mini-Ject®, BioJect®, PowderJect®, VitaJect®, AdvantaJet®, MediJect®, Injex 30®, MHI-500®, LectraJet®, and implaJect®. There are many ways of supplying the energy for the injection, including but not limited to, compressed gas, liquefied gasses such as CO₂, electrical energy, or combustible materials. In the preferred embodiment, the needle free injector is a pre-filled injector wherein the energy for the injection is supplied by a pressurized gas charge, as described within U.S. Pat. Nos. 5,891,086, 6,620,135, and 5,480,381. Needle free injectors can be filled at the time of use, although in the preferred embodiment, the needle free injector is pre-filled. It will be obvious to one skilled in the art that any needle-free injector currently available or developed in the future can be used to carry out the invention. All of the above cited patents are incorporated herein by reference in their entirety and in order to disclose and describe a device for needle-free subcutaneous injections of formulations of the antithrombotic agent enoxaparin.

### Self Administration

In general, patients will receive antithrombotics following surgery. Examples of procedures include, but are not limited to, hip replacement, knee replacement, hip fracture, or abdominal surgery. Although these are in-patient procedures, and the initial dose of antithrombotic will be administered by skilled caregivers, the patient will need additional doses after they are released from the hospital and are recovering in a home setting.

It is a problem that these patients would need to self-administer an injection of an antithrombotic agent with a needle and syringe. Many people have a fear of needles, and do not wish to self-inject with a needle and syringe. Self-injection requires some skill and training is required. The needle has the potential for needle-stick injury, both to the patient and family members and others in the home. Following injection, needles require special disposal procedures and containers, both as sharps and as medical waste, which are not in general found in the home. Thus, an aspect of the invention includes screening patients with respect to all or any of the above e.g. the patient's fear of needles, ability to follow directions, ability to properly dispose of needles.

When the screen patients are found to have one or more of the problems or issues described above it can be handled with a pre-filled needle-free injector. A needle-free injector eliminates the problems of fear, needle-stick injury and disposal. Because there is no needle, needle phobia is elimated. In the preferred embodiment, the needle-free injector is actuated simply by pressing it into the skin, eliminating the need for extensive training. In the preferred embodiment, the needle-free injector is prefilled, eliminating the need for the patient to fill the device. These advantages will lead to better compliance and ultimately to better medical outcomes.

The method of the invention may comprise the steps of:

Screening patients for a number of factors including their fear of needles, their ability to manipulate needles while avoiding sticking themselves or others accidentally; their ability and facilities for disposing of needles safely. After determining that the patient meets one or more of the criteria the patient may be provided with a needleless drug delivery device in combination with active antithrombotic medications. The patient is then instructed with respect to how to use the drug delivery device by placing a portion against the skin and activating the device in order to inject the antithrombotic medication into the patient. The patient may further be provided with instructions regarding dosing and the timing of dosing. For example, patient's scheduled for surgery may be administered a formulation comprising 30 mg to 40 mg of an antithrombotic medication such as enoxaparn. The medication may be administered once a day or twice a day i.e. every twelve to 24 hours for a period of time from one week to four weeks or for 21 days following surgery.

In accordance with the method of the invention it is desireable to screen patients prior to surgery. The patients may be patients which are being screen prior to hip or knee replacement surgery.

In accordance with the methodology of the invention the patient may be matched with particular dosing schedules depending on their surgery and provided schedules which allow for the administration of the drug for longer or shorter periods of time after surgery depending on the type of surgery and the age and condition of the patient.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A device for delivery of an antithrombotic medicament, comprising:
a needle-free injector; and
a liquid formulation comprising a pharmaceutically acceptable, injectable carrier and a pharmaceutically active antithrombotic agent.

2. The device of claim 1, wherein the needle free injector comprises a gas-powered actuator comprising a spring, and a drive member movable through a stroke to cause a substance to be delivered by the injector.

3. The device of claim 1, wherein the active antithrombotic agent is enoxaparin.

4. The device of claim 1, characterized by being pre-filled, single use, and disposable and labeled with a characteristic chosen from, drug name, drug dose amount, day and time.

5. The device of claim 3, wherein the formulation comprises between 10 mgs/mL and 200 mgs/mL of enoxaparin.

6. The device of claim 1, wherein the formulation comprises 30 mgs or 40 mgs of enoxaparin in a liquid solution with a total volume of between 0.5 mL and 2 mL.

7. A kit comprising a plurality of needleless injector devices for delivery of an antithrombotic medication, the kit comprising:
a plurality of single use, disposable needle-free injectors, wherein each injector is loaded with a liquid formulation comprising a pharmaceutically acceptable, injectable carrier and a pharmaceutically active antithrombotic agent.

8. The kit as claimed in claim 7, wherein each needleless injector is labeled with a label chosen from drug name, drug dose amount, day and time.

9. The kit as claimed in claim 7, wherein the kit further comprises instructions for using the needleless injectors.

10. A method of treatment, comprising:
screening a patient with respect to factors relating to injection with a needle including the patient's fear of needles and the patient's ability to use a needle for injection;
providing to a patient which has a fear of needles or an inability to correctly use a needle injector a needle free injector device which provides for needle-free delivery of a liquid formulation comprising an antithrombotic agent; and
instructing the patient with respect to the use of the device.

11. The method of claim 10, wherein the needle free injector comprises a gas-powered actuator comprising a spring, and a drive member movable through a stroke to cause a substance to be delivered by the injector.

12. The method of claim 10, wherein the patient is provided with a plurality of single use, disposable needle-free injectors, wherein each injector is loaded with a liquid formulation comprising a pharmaceutically acceptable, injectable carrier and a pharmaceutically active antithrombotic agent.

13. The method as claimed in claim 10, wherein the patient recently had surgery.

14. The method as claimed in claim 13, wherein the surgery is chosen from hip surgery, knee replacement surgery and abdominal surgery.

15. The method as claimed in claim 12, wherein the patient is a patient suffering from clot formation.

16. The method of claim 10, wherein the patient is chosen from patients about to undergo hip or knee replacement surgery, abdominal surgery, medical patients who are at risk for thromboembolic complications due to severely restricted mobility during acute illness, a patient who requires treatment of an emergent acute venous thromboembolism or deep vein thrombosis while or after clot formation is occurring or has already occurred and the patient is to receive or self-administer by needle-free injection a formulation containing ORG31550 (Organon).

17. The method of claim 10, wherein the active antithrombotic agent is chosen from idraparinux, α-NPAP [α-N-(2-naphtalenesulfonyl)-glycyl-D-4-aminophenylalanyl-piperidine], dalteparin, nadroparin, tinzaparin, ardeparin, reviparin, fondaparinux and danaparoid.
